(19) United States
(12) Patent Application Publication
(10) Pub. No.: US 2009/0324586 A1
(43) Pub. Date: Dec. 31, 2009

(54) LYOPHILIZATION CYCLE ROBUSTNESS STRATEGY

(75) Inventors: Serguei Tchessalov, Andover, MA (US); Daniel Dixon, Arlington, MA (US); Anthony Barry, Winchester, MA (US); Nicholas Warne, Andover, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/492,052

(22) Filed: Jun. 25, 2009

Related U.S. Application Data
(60) Provisional application No. 61/076,129, filed on Jun. 26, 2008.

(51) Int. Cl. A61K 39/395; F26B 5/06; G01N 33/48; A61K 38/16; A61K 38/43

(52) U.S. Cl. 424/130.1; 34/284; 436/86; 514/12; 424/94.1

(57) ABSTRACT

The present invention provides methods for assessing and optimizing lyophilization cycle robustness. In particular, the present invention provides rapid assessment of cycle robustness with respect to a variety of lyophilization process deviations by varying a relatively small number of parameters.

### RELATED APPLICATIONS

This application claims priority to U.S. Provisional Patent Application Ser. No. 61/076,129, filed on Jun. 26, 2008; the entirety of which is hereby incorporated by reference.

### BACKGROUND OF THE INVENTION

Lyophilization or freeze-drying is a process widely used in the pharmaceutical industry for the preservation of biological and pharmaceutical materials. In lyophilization, water present in a material is converted to ice during a freezing step and then removed from the material by direct sublimation under low-pressure conditions during a primary drying step. During freezing, however, not all of the water is transformed to ice. Some portion of the water is trapped in a matrix of solids containing, for example, formulation components and/or the active ingredient. The excess bound water within the matrix can be reduced to a desired level of residual moisture during a secondary drying step. All lyophilization steps, freezing, primary drying and secondary drying, are determinative of the final product properties.

Current lyophilization robustness strategies incorporate either a factorial design of varying parameters or a statistically designed experiment and typically involve 12-15 different cycles where different parameters or combinations of parameters are each varied. This is a very material intensive, time-consuming process.

Typically, lyophilization cycle robustness is a topic reserved for late stage development, validation and support of commercial lyophilization cycles. In contrast, clinical stage materials are manufactured infrequently, and over a shorter time frame. Because of the limited availability of materials and small number of lots manufactured, robustness may be as important for clinical stage products due to the cost to clinical programs of a lost batch. Additionally, the project timelines and material availability for laboratory lyophilization cycle assessment prefer a targeted approach to robustness.

### SUMMARY OF THE INVENTION

The present invention provides novel and inventive approaches for assessing lyophilization cycle robustness. In particular, the present invention provides rapid assessment of lyophilization cycle robustness to a wide variety of process deviations by only varying a small number of parameters (e.g., two parameters) and monitoring product reaction to these variations. Thus, the present invention provides a significant improvement and advantages over the existing time-consuming and material intensive methods, especially, for early stage clinical products.

In one aspect, the present invention provides methods for assessing lyophilization cycle robustness including steps of: (1) determining a control cycle; (2) executing a number of deviation-driven cycles, wherein the number of deviation-driven cycles is less than 9 (e.g., 1, 2, 3, 4, 5, 6, 7, 8 cycles); (3) comparing lyophilized product from each of the executed deviation-driven cycle to that of the control cycle; and (4) assessing the lyophilization cycle robustness based on the comparison result from step (3).

In some embodiments, step (1) includes optimizing the control lyophilization cycle. In some embodiments, the number of deviation-driven cycles is 2.

In some embodiments, step (3) includes comparing a degradation rate of the lyophilized product. In some embodiments, the degradation rate is determined by a stability indicating assay. In some embodiments, the degradation rate is determined by Size Exclusion HPLC (SE-HPLC).

In some embodiments, step (3) includes comparing the cake quality of the lyophilized product. In some embodiments, the cake quality is determined by moisture measurement and/or powder modulated differential scanning calorimetery (MDSC).

In some embodiments, the deviation-driven cycles are designed to vary one or more product parameters. In some embodiments, the one or more product parameters include a product temperature. In some embodiments, the one or more product parameters include a product residual moisture.

In some embodiments, the deviation-driven cycles include cycles with deviations from programmable cycle parameters selected from the group consisting of shelf temperature, pressure, drying time, and combinations thereof.

In some embodiments, the deviation-driven cycles include cycles with deviations from parameters selected from the group consisting of increase in shelf temperature or pressure during primary drying, incomplete primary drying hold due to decrease in shelf temperature, pressure or time, shortened secondary drying time, secondary drying with decreased shelf temperature, and combinations thereof.

In some embodiments, the deviation-driven cycles include a cycle with increased shelf temperature or pressure during primary drying to increase the product temperature as compared to the control cycle. In some embodiments, the increased product temperature during primary drying is 4-10° C. above optimized product temperature during primary drying in the control cycle.

In some embodiments, the deviation-driven cycles include a cycle with modified or significantly altered primary drying step. In some embodiments, the deviation-driven cycles include a cycle with primary drying performed at the same temperature as a secondary drying step. In some embodiments, the deviation-driven cycles include a cycle omitting primary drying.

In some embodiments, the deviation-driven cycles include a cycle with increased residual moisture as compared to the control cycle. In some embodiments, the increased residual moisture ranges from 1.2-4.5% moisture. In some embodiments, the increased residual moisture ranges from 1.5-3% moisture. In some embodiments, the control cycle includes 0-2% residual moisture. In some embodiments, the control cycle includes 0-1% residual moisture.

In some embodiments, the deviation-driven cycles include a cycle with shortened secondary drying time. In some embodiments, the deviation-driven cycles include a cycle omitting secondary drying hold. In some embodiments, the deviation-driven cycles include a cycle with stoppering at the completion of primary drying. In some embodiments, the deviation-driven cycles include a cycle with decreased shelf temperature during secondary drying.

In some embodiments, inventive methods in accordance with the present invention are developed to lyophilize proteins. In some embodiments, suitable proteins include antibodies (e.g., monoclonal antibodies) or fragments thereof, growth factors, clotting factors, cytokines, fusion proteins, pharmaceutical drug substances, vaccines, enzymes, Small Modular ImmunoPharmaceutical™ (SMIP™) proteins). In some embodiments, inventive methods in accordance with the present invention are developed to lyophilize antibodies or antibody fragments including, but not limited to, intact IgG, F(ab′)2, F(ab)2, Fab′, Fab, ScFv, single domain antibodies (e.g., shark single domain antibodies (e.g., IgNAR or fragments thereof)), diabodies, triabodies, tetrabodies. In some embodiments, inventive methods are developed to lyophilize monoclonal antibodies. Inventive methods in accordance with the present invention can also be developed for nucleic acids (e.g., RNAs, DNAs, or RNA/DNA hybrids, aptamers), chemical compounds, small molecules, natural products, to name but a few.

In some embodiments, the present invention provides methods of determining a lyophilization cycle for production including a step of assessing the lyophilization cycle robustness using various methods described herein.

In some embodiments, the present invention provides methods of producing lyophilized products including executing a lyophilization cycle assessed by various methods described herein.

In some embodiments, the present invention provides methods of providing lyophilized product for, e.g., an early clinical stage process including executing a lyophilization cycle assessed by various methods described herein.

In addition, inventive methods in accordance with the present invention can be used to evaluate potential product impact of process deviations during manufacturing. Inventive methods in accordance with the present invention can also be used to evaluate lyophilization equipment for product manufacturing.

The present invention further provides lyophilized pharmaceutical products produced using a lyophilization cycle assessed by methods in accordance with the present invention.

As used in this application, the terms “about” and “approximately” are used as equivalents. Any numerals used in this application with or without about/approximately are meant to cover any normal fluctuations appreciated by one of ordinary skill in the relevant art. For example, normal fluctuations of a value of interest may include a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are for illustration purposes only, not for limitation.

FIG. 1 illustrates an exemplary control lyophilization cycle.

FIG. 2 illustrates an exemplary aggressive lyophilization cycle.

FIG. 3 illustrate an exemplary comparison of primary drying temperature profiles between aggressive and control lyophilization cycles.

FIG. 4 illustrates visual appearance of exemplary lyophilized cakes where aggressive cycle remained below collapse temperature.

FIG. 5 illustrate exemplary comparison of cake appearance for lyophilized Molecule I: partial collapse (aggressive cycle, left vial) versus intact cake (baseline cycle, right vial).

FIG. 6 illustrates an exemplary high moisture lyophilization cycle.

FIG. 7 illustrates comparison of exemplary primary drying temperature profiles between high moisture and control lyophilization cycles.

FIG. 8 illustrates exemplary thermocouple overlay and predicted temperatures.

### DETAILED DESCRIPTION OF THE INVENTION

The present invention provides novel and inventive methods for assessing lyophilization cycle robustness. Among other things, the present invention provides rapid assessment of cycle robustness to a wide variety of process deviations by only varying a small number of parameters (e.g., two parameters) and monitoring product reaction to these variations.

In some embodiments, the invention provides methods for assessing lyophilization cycle robustness by effectively designing and executing a small number of deviation-driven cycles and comparing lyophilized products from each deviation-driven cycle to that from a suitable control or target cycle.

As used herein, a control cycle represents the target cycle for scale-up to manufacturing. Typically, stability has been established for material lyophilized using the control or target cycle. For example, a control cycle is typically designed to produce stable lyophilization of the product well below certain temperature (e.g., collapse temperature).

Typically, a control cycle is designed in a laboratory to optimize the freeze-drying process. In some embodiments, an optimum freeze-drying process is a process that achieves the highest protein stability for the least cost. Generally, defining a control cycle involves optimization of various controllable stages of freeze-drying, including, freezing, primary drying, and secondary drying. For example, defining a control cycle involves determining optimum cooling rate, freezing temperature and time, target product temperature, chamber pressure, shelf temperature, secondary drying heating conditions including heating rate and chamber pressure, the shelf temperature and secondary drying time, and residual moisture. A suitable control cycle for a particular material of interest can be determined by various methods known in the art. For example, exemplary methods and principles are described in Tang et al. (2004) “Design of Freeze-Drying Processes for Pharmaceuticals: Practical Advice,” Pharmaceutical Research, 21:191-200, the contents of which are hereby incorporated by reference herein.

Scale-up of the control cycle for manufacturing production would not provide for any process tolerance or variability. During lyophilization, process parameters occasionally deviate from the set point, sometimes for significant periods of time. Thus, deviation-driven cycles are developed to assess the impact of such deviations in the lyophilization process and the product. As used herein, the term “deviation-driven cycles” refers to any lyophilization cycle designed to vary one or more parameters of the product or the freeze-drying process. In some embodiments, deviation-driven cycles are developed to vary one or more product parameters including, but not limited to, product temperatures, or product residual moisture. In some embodiments, deviation-driven cycles are developed to vary programmable cycle parameters including, but not limited to, shelf temperature, pressure (e.g., chamber pressure), drying time, primary drying end point, sublimation rate, secondary drying conditions (e.g., heating rate, chamber pressure, shelf temperature, drying time).

In some embodiments, a suitable deviation-driven cycle may have increased shelf temperature or pressure during primary drying. In some embodiments, a suitable deviation-driven cycle may have incomplete primary drying hold due to decrease in shelf temperature, pressure or time. In some embodiments, a suitable deviation-driven cycle may have shortened secondary drying time, or secondary drying with decreased shelf temperature. In some embodiments, a suitable deviation-driven cycle may have increased shelf temperature or pressure during primary drying to increase the product temperature as compared to the control cycle (e.g., 4-10° C. above optimized product temperature during primary drying in the control cycle). In some embodiments, a deviation-driven cycle may have modified or significantly altered primary drying step. For example, a deviation-driven cycle may have a primary drying performed at the same temperature as a secondary drying step. In some embodiments, a deviation-driven cycle may omit primary drying altogether.

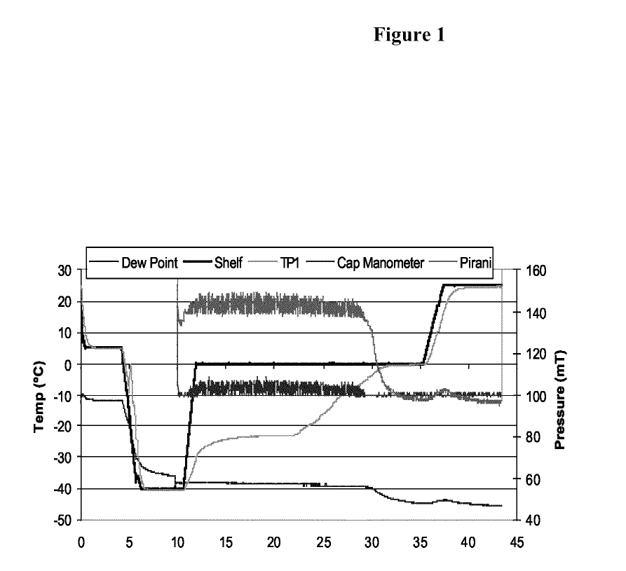

As non-limiting examples described in the Examples section, the present invention was able to assess lyophilization robustness by executing three cycles, e.g., (1) control cycle, (2) aggressive drying cycle, and (3) elevated moisture cycle. The aggressive cycle performed all drying at 25-30° C., 100 mTorr process conditions, the target secondary condition. The elevated moisture cycle was stoppered at the conclusion of primary drying (0° C. shelf temperature) to generate a moisture result well above that observed in the control cycle. Examples of the three cycles are shown in FIGS. 1, 2 and 6. The aggressive cycle omits the primary drying hold and performs all lyophilization under the secondary drying conditions. This results in much faster, higher temperature drying. By omitting the secondary drying hold, the high moisture cycle yields material that is at elevated moisture, and of higher moisture than the anticipated larger scale manufacturing cycle.

Typically, lyophilized product can be assessed based on cake quality and appearance, product quality analysis, reconstitution time, quality of reconstitution, high molecular weight, moisture, and glass transition temperature. For example, cake quality analysis includes moisture measurement and powder mDSC. Typically, product quality analysis includes product degradation rate analysis using methods including, but not limited to, size exclusion HPLC (SE-HPLC), cation exchange-HPLC (CEX-HPLC), X-ray diffraction (XRD), modulated differential scanning calorimetry (mDSC) and other means known to one of skill in the art.

Inventive methods in accordance with the present invention can be utilized to assess any lyophilization cycles developed for any materials including, but not limited to, proteins, peptides, nucleic acids (e.g., RNAs, DNAs, or RNA/DNA hybrids, aptamers), chemical compounds, small molecules, drug substances, natural products. In some embodiments, the present invention is utilized to assess or determine lyophilization cycles suitable for proteins including, but not limited to, antibodies (e.g., monoclonal antibodies) or fragments thereof, growth factors, clotting factors, cytokines, fusion proteins, pharmaceutical drug substances, vaccines, enzymes, Small Modular ImmunoPharmaceuticals™ (SMIPs). In some embodiments, the present invention is utilized to assess or determine lyophilization cycles suitable for antibodies or antibody fragments including, but not limited to, intact IgG, F(ab′)2, F(ab)2, Fab′, Fab, ScFv, single domain antibodies (e.g., shark single domain antibodies (e.g., IgNAR or fragments thereof)), diabodies, triabodies, tetrabodies.

Typically, materials to be lyophilized are prepared in liquid formulations. In some embodiments, inventive methods in accordance with the present invention are developed for protein formulations. In some embodiments, suitable protein formulations contain a protein of interest at a concentration in the range of about 1 μg/ml to 150 mg/ml (e.g., about 1 μg/ml to 100 μg/ml, about 1 μg/ml to 1 mg/ml, about 25 μg/ml to 1 mg/ml, about 25 μg/ml to 50 mg/ml, about 1 mg/ml to 25 mg/ml, about 1 mg/ml to 50 mg/ml, 1 mg/ml to 75 mg/ml, 1 mg/ml to 100 mg/ml). In some embodiments, suitable protein formulations contain a protein of interest at a concentration of about 1 μg/ml, about 25 μg/ml, about 50 μg/ml, about 75 μg/ml, about 100 μg/ml, about 150 μg/ml, about 200 μg/ml, about 250 μg/ml, about 500 μg/ml, about 1 mg/ml, about 10 mg/ml, about 20 mg/ml, about 30 mg/ml, about 40 mg/ml, about 50 mg/ml, about 75 mg/ml, about 100 mg/ml, about 150 mg/ml. In some embodiments, a suitable protein formulation contains a bulking agent selected from the group consisting of sucrose, glycine, sodium chloride, lactose and mannitol, a stabilizer selected from the group consisting of sucrose, trehalose, arginine, and sorbitol, and/or a buffer selected from the group consisting of tris, histidine, citrate, acetate, phosphate and succinate.

Lyophilization may be performed in a container, such as a tube, a bag, a bottle, a tray, a vial (e.g., a glass vial) or any other suitable containers. The containers may be disposable. Controlled freeze and/or thaw may also be performed in a large scale or small scale.

Inventive methods in accordance with the present invention can be used to assess lyophilization cycles developed for various lyophilizers, such as, commercial-scale lyophilizers, pilot-scale lyophilizers, or laboratory-scale lyophilizers.

It should be understood that the above-described embodiments and the following examples are given by way of illustration, not limitation. Lyophilization cycle robustness strategy in accordance with the present invention can be applied to any molecules (e.g., proteins) in general. For example, the molecules A-I used in the following examples can be any proteins, antibodies, nucleic acids, chemical compounds, vaccines, enzymes, small molecules, or any other types of molecules. Various changes and modifications within the scope of the present invention will become apparent to those skilled in the art from the present description.

### EXAMPLES

### Example 1

### Formulations and Product Assessment

Exemplary formulations, in pre-lyophilization liquid state, contain about 10 mM histidine, 5% sucrose, +/−10 mM Methionine, +/−0.01% Polysorbate-80, and about 50 mg/mL candidate proteins. The liquid formulations were distributed to suitable container/closure system. In this example, 5 mL West tubing vials (rinsed and autoclaved) with West 20 mm lyophilization stoppers (autoclaved and dried) were used.

Lyophilized product assessment includes two types of analysis—cake quality and product quality. Cake quality included moisture measurement and powder MDSC. The primary product quality assay was SE-HPLC, which has been shown to be the most sensitive stability-indicating assay for these lyophilized products. Additionally, product thermocouple data was analyzed using a heat transfer model to assess the cake resistance to mass transfer, and the anticipated product temperature profiles within the pilot-scale lyophilizer. For all proteins tested, high molecular weight generation was the most stability indicating assay product, and thus was monitored as a function of storage. Moisture post lyophilization was measured by Karl Fischer titration. Differential Scanning Calorimetry-Q1000 (TA Instruments, New Castle, Del.) was used for sub ambient and powder glass transition temperature determination in modulated mode.

Linkam cold stage, utilizing Pax-IT image collection software, was used to perform freeze-drying microscopy. Freezing protocol mimicked the lyophilization freezing profile. During sublimation, temperature set points were maintained for a minimum of 15 minutes before advancing to next set point.

Frozen product was assessed by MDSC. These formulations typically showed two glass transitions—one measured at −28° C. to −25° C. and one often observed between −12° C. and −8° C. By Freeze Drying Microscopy, a collapse is usually observed in the temperature range −18° C. to −15° C.

### Example 2

### Defining the Control Cycle

A target cycle has been defined that results in lyophilization of the product well below the critical (often collapse) temperature. An example of this cycle is shown in FIG. 1. Analytical results are shown in Table 1 for initial (moisture and glass transition temperature, Tg) and accelerated storage conditions (high molecular weight increase, to monitor the primary route of degradation for these lyophilized products). Additional product characteristics that were monitored after lyophilization, samples were examined for cake appearance, reconstitution time, and quality of reconstitution. Secondary structure assessment via FTIR was also performed on some of the t=0 samples.

In all of these instances, suitable storage stability has been demonstrated under refrigerated conditions (the recommended storage temperature). The use of 50° C. (40° C. for proteins H and I) as an accelerated storage condition is to observe degradation more rapidly for comparison purposes. Based on the established, acceptable stability profile, the product temperature profile during the control cycle is the target for scale-up to manufacturing production.

TABLE 1
Analytical results for Control cycle.

| | Initial Moisture | Initial Tg | delta % HMW (4 wks/ |
|---|---|---|---|
| Molecule | (%) control | (° C.) | 50° C.) control |
| A | 0.5% | 86 | 0.17% |
| B | 0.7% | 87 | 0.7% |
| C | 0.4% | 85 | 0.7% |
| D | 0.6% | 84 | n/d |
| E | 0.4% | 91 | 1.2% |
| | | 64 | |
| F | 0.8% | 86 | 0.3% |
| G | 0.7% | 84 | 0.8% |
| | | | All data below collected |
| | | | at 3 mo/40° C. |
| H | 0.4% | 89 | 0.5% |
| I | 0.3% | 94 | 0.7% |
| | | 48 | |

### Example 3

### Assessing an Aggressive Condition

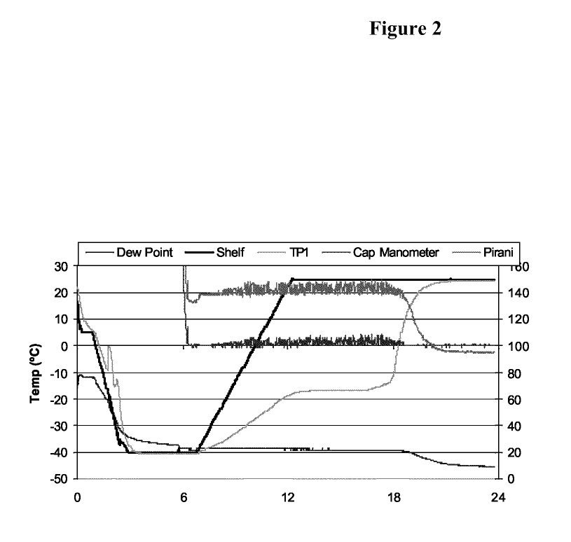

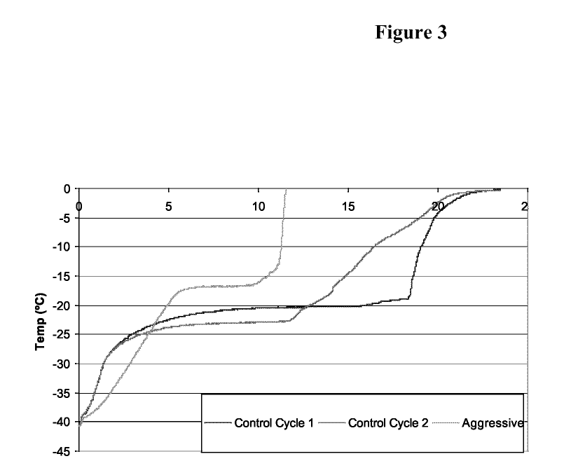

The purpose of the aggressive cycle is to significantly increase the product temperature during primary drying by increasing the shelf temperature to the secondary drying set point. An example of this is shown in FIG. 2. FIG. 3 shows the difference between the primary drying temperature profile of material lyophilized in two different control cycles (pink and blue lines) and from the aggressive cycle (green line). In this case, the aggressive cycle lead to an increase in product temperature of 5-7° C. over the control cycle.

The analytical results comparing the product from the aggressive cycle with control material is shown in Table 2. All molecules showed comparable results regarding initial moisture, initial glass transition temperature, and high molecular weight increase over accelerated temperature storage. The comparable stability profile between material lyophilized with the aggressive and control cycles defines a suitable design space for product temperatures during primary drying.

TABLE 2
Analytical comparison between control and aggressive cycles.

| | | | | | delta % HMW | |
|---|---|---|---|---|---|---|
| | | | | | (4 wks/50° C.) | |
| | Initial Moisture (%) | | Initial Tg (° C.) | | | aggres- |
| Molecule | control | aggressive | control | aggressive | control | sive |
| A | 0.5% | 0.5% | 86 | 85 | 0.17% | 0.16% |
| B | 0.7% | 0.7% | 87 | 86 | 0.7% | 0.7% |
| C | 0.4% | 0.7% | 85 | 85 | 0.7% | 0.7% |
| D | 0.6% | 0.6% | 84 | 76 | n/d | −0.3% |
| E | 0.4% | 0.2% | 91 | 95 | 1.2% | 1.2% |
| | | | 64 | 59 | | |
| F | 0.8% | 0.2% | 86 | 80 | 0.3% | 0.6% |
| G | 0.7% | 0.5% | 84 | 86 | 0.8% | 1.2% |
| | | | | | All data below | |
| | | | | | collected at | |
| | | | | | 3 mo/40° C. | |
| H | 0.4% | 0.4% | 89 | 88 | 0.5% | 0.5% |
| I | 0.3% | 0.3% | 94 | 73 | 0.7% | 0.7% |
| | | | 48 | 45 | | |

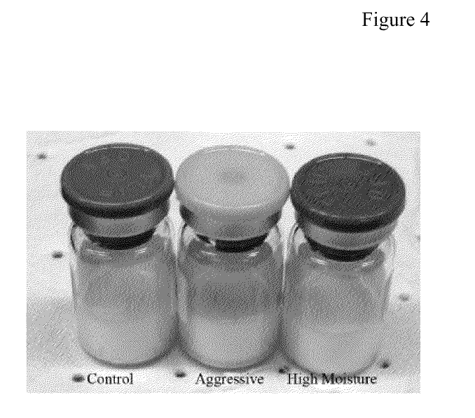

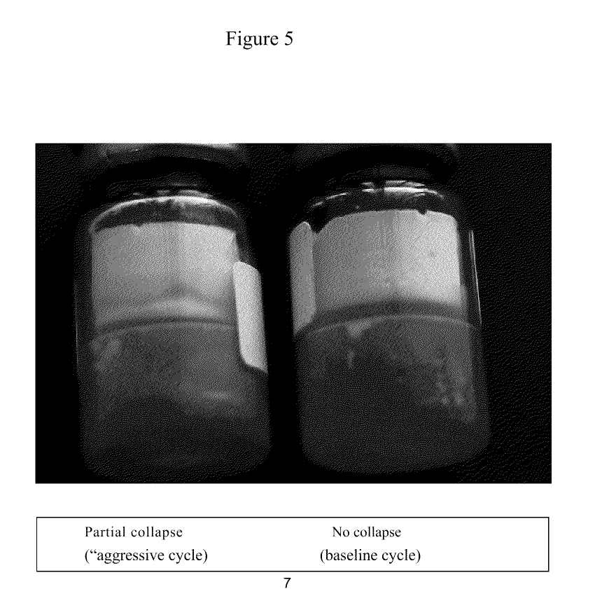

Aggressive drying in this strategy resulted in some products being lyophilized slightly below the measured collapse temperature, and some slightly above. FIG. 4 shows one example (A in Table 2) where the product remained below the collapse temperature during the aggressive cycle and there is no visual evidence of collapse. In contrast, FIG. 5 shows a case where partial collapse was observed at the bottom of the vial during the aggressive cycle for 1. In this case, the product slightly exceeded the collapse temperature during lyophilization, however the moisture and high molecular weight profiles were identical to control.

### Example 4

### Assessing Elevated Residual Moisture

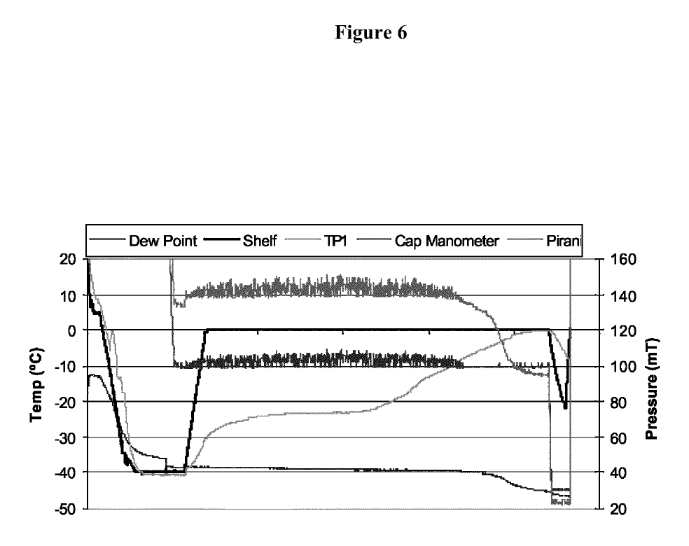

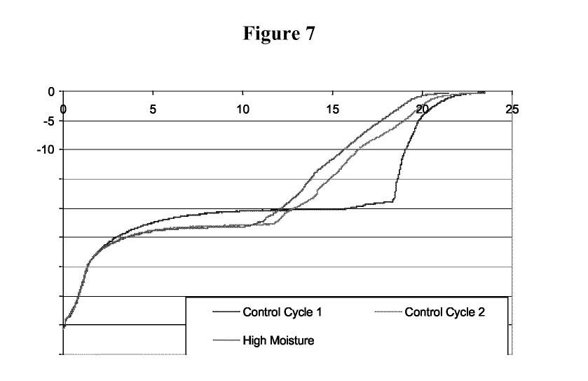

One frequently referenced consequence of collapse (whether macroscopic or microscopic) is an increase in residual moisture. To assess the compatibility of products with moistures significantly higher than those generated by the target or aggressive cycles, a third cycle was executed which was truncated at the conclusion of primary drying (FIG. 6). Up to the conclusion of primary drying, all cycle parameters were identical to the control cycle, and as a result, the product thermocouple profile was also comparable between high moisture (brown line) and control cycles (FIG. 7).

### Example 5

### Post Lyophilization Assessment

After lyophilization, samples were examined for cake appearance, reconstitution time, quality of reconstitution, high molecular weight, moisture, and glass transition temperature. Secondary structure assessment via FTIR was also performed on some of the t=0 samples.

In addition to analysis immediately after lyophilization, this material was enrolled into the same high temperature storage study as materials from the previous two cycles. The results of this data are summarized in Table 3. As expected, all of these materials had significantly higher moisture results than materials from control and aggressive cycles, which lead to a decrease in the dry powder glass transition temperature. In nearly all instances, the glass transition temperature of the high moisture material was within 15° C. of the storage condition without detrimental stability results.

TABLE 3
Product results for 9 proteins utilizing robustness strategy.

| | Initial moisture, % | | | Initial Tg, ° C. | | | Delta HMW(%), 4 wks at 50° C. | | |
|---|---|---|---|---|---|---|---|---|---|
| | | | High | | | High | | | High |
| Molecule | Control | Aggressive | moisture | Control | Aggressive | moisture | Control | Aggressive | moisture |
| A | 0.5% | 0.5% | 2.9% | 86 | 85 | 56 | 0.17% | 0.16% | 0.11 |
| B | 0.7% | 0.7% | 2.8% | 87 | 86 | 56 | 0.7% | 0.7% | 0.9% |
| C | 0.4% | 0.7% | 2.5% | 85 | 85 | 62 | 0.7% | 0.7% | 0.5% |
| D | 0.6% | 0.6% | 2.6% | 84 | 76 | 48 | n/d | −0.3% | 0.6% |
| E | 0.4% | 0.2% | 1.9% | 91 | 95 | 69 | 1.2% | 1.2% | 0.8% |
| | | | | 64 | 59 | 44 | | | |
| F | 0.8% | 0.2% | 1.5% | 86 | 80 | 76 | 0.3% | 0.6% | 0.3% |
| G | 0.7% | 0.5% | n/d | 84 | 86 | n/d | 0.8% | 1.2% | n/d |
| | | | | | | | All data below collected at 3 | | |
| | | | | | | | mo/40° C. | | |
| H | 0.4% | 0.4% | 1.3% | 89 | 88 | 43 | 0.5% | 0.5% | 0.3% |
| I | 0.3% | 0.3% | 1.4% | 94 | 73 | 98 | 0.7% | 0.7% | 0.4% |
| | | | | 48 | 45 | 55 | | | |

The resulting materials were compared over a short term, high temperature stability study. The 9 candidate proteins examined have all shown comparable degradation between the three cycles over the duration of the stability cycles. High molecular weight generation is the most significant degradation product for the candidate proteins during storage. Table 1 shows the initial moisture and glass transition values, as well as the increase in high molecular weight after 4 weeks at 50° C. (or three months at 40° C., in the case of proteins H and I).

### Example 6

### Scale-Up

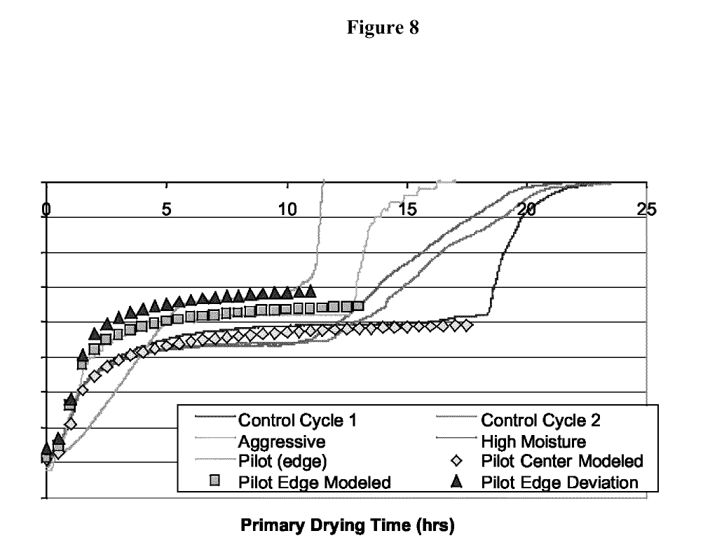

The Pilot scale lyophilizer used in the manufacture of these protein molecules has been previously characterized, in terms of heat transfer and sublimation capacity (see, e.g., Tchessalov, Dixon, Warne. 2007. Principles of Lyophilization Scale-Up. American Pharmaceutical Review 10(3):88-92). Additionally, the impact of additional super-cooling, due to a lower particulate environment, on the resistance to mass transfer has been quantified (as a worst case estimate). This information has allowed for the application of a simplified heat and mass transfer model to define the suitable Pilot lyophilization cycle, and estimate process tolerances (see, e.g., Pikal. 1985. Use of Laboratory Data in Freeze Drying Process Design Heat and Mass Transfer Coefficients and the Computer Simulation of Freeze Drying. Journal of Parenteral Science and Technology 39(3):115-138). Based on the results from the laboratory-scale studies (providing product information) and lyophilizer characterization (providing equipment limitations), an operating space can be defined in the Pilot scale lyophilizer, using Quality by Design principles (see, e.g., Nail, Searles. 2008. Elements of Quality by Design in Development and Scale-Up of Freeze-Dried Parenterals. BioPharm International: 44-52). FIG. 8 shows primary drying thermocouple traces of 4 laboratory cycles (blue, pink, teal, and green lines), one Pilot cycle (orange line), and modeled results.

Based on the increase in cake resistance and measured heat transfer coefficient, the anticipated edge vial temperature profile (representative of measured thermocouples) is shown as blue squares above. These results agree very closely with the measured orange thermocouple data. Adjusting the heat transfer for Pilot center vials yields the yellow diamonds, which agrees very well with laboratory thermocouple data (used to generate preliminary stability assessments) (see, e.g., Tchessalov, Warne. 2008. Lyophilization: cycle robustness and process tolerances, transfer and scale up. European Pharmaceutical Review (3):76-83).

The purple diamonds represented the calculated Pilot edge thermocouple profile in the event of a primary drying deviation of +5° C. shelf temperature and +20 mTorr chamber pressure. This deviation is outside of allowable process tolerances, and beyond anything observed in over 9 years of manufacturing experience. This worst-case product temperature data agrees very well with the thermocouple profile of the aggressive cycle.

### Example 7

### Process Deviation Application

The following exemplary types of deviations can be readily covered by this strategy:
1. Increase in shelf temperature or pressure during primary drying (e.g., aggressive cycle).
2. Incomplete primary drying hold due to decrease in shelf temperature, pressure or time (e.g., aggressive cycle performed primary drying at secondary drying condition, or omitting hold for primary drying completely).
3. Secondary drying time ended too early, or shelf temperature didn't reach target (e.g., high moisture cycle omitted secondary drying hold completely).
4. Transient/short duration occurrences of any of the above.

These deviations are addressed from a product temperature perspective. By identifying the impact of a process deviation on product temperature, the appropriate product quality assessment can be established based on (1) modeling results, and (2) comparison with the two executed deviation-driven cycles. Because the above conditions encompass all observed manufacturing process deviations, lyophilization cycle deviations have not lead to the loss or delayed release of a single batch.

For all molecules tested, stability profile comparable between all cycles after 4 weeks at 50° C. This helps justify not only process deviations, but also moisture specification. Major glass transition temperatures well above storage temperature for all cycles. The glass transition temperatures of high moisture samples was lower than others, as expected, due to increased water content. High moisture samples had glass transition temperatures that were occasionally close to storage temperature, but this did not appear to significantly impact stability. Product temperature perturbations during aggressive cycle were far greater than deviations expected during manufacturing, providing product quality justification in the event of manufacturing deviations. Strategies in accordance with the present invention have been executed for 9 different molecules so far, with consistent results. These strategies have been used to assess manufacturing deviations that have occurred in the clinical production of these candidate proteins.

### EQUIVALENTS

The foregoing has been a description of certain non-limiting embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects is excluded are not set forth explicitly herein.

### INCORPORATION BY REFERENCE

All publications and patent documents cited in this application are incorporated by reference in their entirety for all purposes to the same extent as if the contents of each individual publication or patent document were incorporated herein.

What is claimed is:

1. A method for assessing lyophilization cycle robustness comprising steps of:
(1) determining a control cycle;
(2) executing a number of deviation-driven cycles, wherein the number of deviation-driven cycles is less than 9;
(3) comparing lyophilized product from each of the executed deviation-driven cycle to that of the control cycle; and
(4) assessing the lyophilization cycle robustness based on the comparison result from step (3).

2. The method of claim 1, wherein step (3) comprises comparing a degradation rate of the lyophilized product.

3. The method of claim 2, wherein the degradation rate is determined by a stability indicating assay.

4. The method of claim 3, wherein the degradation rate is determined by Size Exclusion HPLC (SE-HPLC).

5. The method of claim 1, wherein step (3) comprises comparing the cake quality of the lyophilized product.

6. The method of claim 5, wherein the cake quality is determined by moisture measurement and/or powder modulated differential scanning calorimetery (MDSC).

7. The method of claim 1, wherein step (1) comprises optimizing the control lyophilization cycle.

8. The method of claim 1, wherein the number of deviation-driven cycles is 2.

9. The method of claim 1, wherein the deviation-driven cycles are designed to vary one or more product parameters.

10. The method of claim 9, wherein the one or more product parameters comprise a product temperature.

11. The method of claim 10, wherein the one or more product parameters comprise a product residual moisture.

12. The method of claim 1, wherein the deviation-driven cycles comprise cycles with deviations from programmable cycle parameters selected from the group consisting of shelf temperature, pressure, drying time, and combinations thereof.

13. The method of claim 1, wherein the deviation-driven cycles comprise cycles with deviations from parameters selected from the group consisting of increase in shelf temperature or pressure during primary drying, incomplete primary drying hold due to decrease in shelf temperature, pressure or time, shortened secondary drying time, secondary drying with decreased shelf temperature, and combinations thereof.

14. The method of claim 1, wherein the deviation-driven cycles comprise a cycle with increased shelf temperature or pressure during primary drying to increase the product temperature as compared to the control cycle.

15. The method of claim 14, wherein the increased product temperature during primary drying is 4-10° C. above optimized product temperature during primary drying in the control cycle.

16. The method of claim 1, wherein the deviation-driven cycles comprise a cycle with modified or significantly altered primary drying step.

17. The method of claim 16, wherein the deviation-driven cycles comprises a cycle with primary drying performed at the same temperature as a secondary drying step.

18. The method of claim 16, wherein the deviation-driven cycles comprises a cycle omitting primary drying.

19. The method of claim 1, wherein the deviation-driven cycles comprise a cycle with increased residual moisture as compared to the control cycle.

20. The method of claim 16, wherein the increased residual moisture ranges from 1.2-4.5% moisture.

21. The method of claim 20, wherein the increased residual moisture ranges from 1.5-3% moisture.

22. The method of claim 19, wherein the control cycle comprises 0-2% residual moisture.

23. The method of claim 19, wherein the control cycle comprises 0-1% residual moisture.

24. The method of claim 19, wherein the deviation-driven cycles comprise a cycle with shortened secondary drying time.

25. The method of claim 24, wherein the deviation-driven cycles comprise a cycle omitting secondary drying hold.

26. The method of claim 25, wherein the deviation-driven cycles comprise a cycle with stoppering at the completion of primary drying.

27. The method of claim 19, wherein the deviation-driven cycles comprise a cycle with decreased shelf temperature during secondary drying.

28. The method of claim 1, wherein the lyophilization cycle is developed for a protein.

29. The method of claim 28, wherein the protein is an antibody or a fragment thereof, a growth factor, a clotting factor, a cytokine, a fusion protein, a pharmaceutical drug substance, a vaccine, an enzyme or a Small Modular ImmunoPharmaceutical (SMIP™).

30. The method of claim 29, wherein the antibody is a monoclonal antibody or a single-domain antibody.

31. A method of determining a lyophilization cycle for production comprising a step of assessing the lyophilization cycle robustness using the method of claim 1.

32. A method of producing a lyophilized product comprising executing a lyophilization cycle assessed by the method of claim 1.

33. A method of providing a lyophilized product for an early clinical stage process, the method comprising executing a lyophilization cycle assessed by the method of claim 1.

34. A method of evaluating potential product impact of process deviations during manufacturing using the method of claim 1.

35. A method of evaluating a lyophilization equipment for product manufacturing using the method of claim 1.

36. A lyophilized pharmaceutical product produced using a lyophilization cycle assessed by the method of claim 1.
